(19) United States
(12) Patent Application Publication
(10) Pub. No.: US 2024/0335114 A1
(43) Pub. Date: Oct. 10, 2024

(54) SYSTEM AND METHOD FOR PREDICTING ILLNESS, DEATH AND/OR OTHER ABNORMAL CONDITION OF AN ANIMAL

(71) Applicant: INTERVET INC., Madison, NJ (US)

(72) Inventors: Aaron MATHANKERI, Madison, NJ (US); Brian Scott SCHUPBACH, Madison, NJ (US); Christopher Eugene LARSEN, Madison, NJ (US)

(21) Appl. No.: 18/290,819

(22) PCT Filed: Jul. 25, 2022
(86) PCT No.: PCT/US2022/038112

§ 371 Date: Jan. 22, 2024

Related U.S. Application Data
(60) Provisional application No. 63/225,574, filed on Jul. 26, 2021.

(51) Int. Cl. A61B 5/00; A61B 5/01; G16H 40/67; G16H 50/20; G16H 50/30; G16H 50/70

(52) U.S. Cl. CPC A61B 5/0008; A61B 5/01; A61B 5/746; G16H 40/67; G16H 50/20; G16H 50/30; G16H 50/70; A61B 2503/40

(57) ABSTRACT

A system and method for predicting an illness, death or other abnormal condition of an animal is disclosed. A record for the animal including a monitored temperature time series of monitored temperature values that are indicative of a temperature of the animal over a given time period is provided. The monitored temperature time-series is a temperature pattern that includes two or more cycles that are defined by a distance between a given peak of the temperature pattern and a successive peak of the temperature pattern, or a distance between a given valley of the temperature pattern and a successive valley of the temperature pattern. The monitored temperature time-series is analyzed. Based on the analysis, a prediction of the illness, death or other abnormal condition of the animal within a given time duration of the given time period is made. A user of the system is notified of the prediction.

### TECHNICAL FIELD

The invention relates to a system and method for predicting illness, death and/or other abnormal condition of an animal.

### BACKGROUND

Currently, it is not possible to accurately predict a given time window within which an animal, for example a cow in a feed lot, is expected to die, in the absence of clearly visible signs or other indicators that the animal is approaching death.

It is an object of the present disclosure to predict an illness, death and/or other abnormal condition of an animal with a high accuracy at an early stage, prior to observing clearly visible signs or other indicators that the animal is approaching death or on the verge of becoming ill or developing an abnormal condition. Actions that can be taken based on this prediction can be, for example, modifying a treatment plan for the animal or developing new treatment options for the animal to prevent or mitigate illness of the animal, prevent death of the animal, or extend the life of the animal: reducing outlays for the animal (e.g., reduce a feed of a feed lot animal), or selling the animal (e.g., a feed lot animal).

Thus, there is a need in the art for a new system and method for predicting illness, death and/or other abnormal condition of an animal.

### GENERAL DESCRIPTION

In accordance with a first aspect of the presently disclosed subject matter, there is provided a system for predicting an illness, death or other abnormal condition of a monitored animal, the system comprising a processing circuitry configured to: provide a monitored record for the monitored animal, the monitored record including a monitored temperature time series of monitored temperature values that are indicative of a temperature of the monitored animal over a given time period: analyze the monitored temperature time-series: predict the illness, death or other abnormal condition of the monitored animal within a given time duration of the given time period, based on the analysis, wherein the monitored temperature time-series is a temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley; and notify a user of the system of the prediction.

In some cases, the processing circuitry is configured to analyze the monitored temperature time series using a Machine Learning (ML) model, the ML model being trained based on a data repository of historical records for a plurality of animals, each historical record of the historical records including: (A) a historical temperature time series of historical temperature values that are indicative of the temperature of a respective animal of the plurality of animals over an earlier time period, being earlier than and of an identical duration to the given time period, and (B) a target field that indicates whether the respective animal became ill, died, or developed any other abnormal condition within the given time duration of the earlier time period.

In some cases, the ML model is a Switching Autoregressive Hidden Markov Model (SAR-HMM).

In some cases, for each cycle of the cycles, a temperature difference between a peak temperature value of the monitored temperature values in the respective cycle and a valley temperature value of the monitored temperature values in the respective cycle is greater than or equal to a predetermined difference.

In some cases, the predetermined difference is at least 4° C.

In some cases, the given time period is two to five days.

In some cases, the given time duration is two months or less.

In some cases, the monitored temperature values are based on raw temperature readings that are read by one or more temperature sensors that are mounted on an animal tag assembly that is placed on the monitored animal.

In some cases, the processing circuitry is further configured to: determine, for each cycle of the cycles, whether the peak temperature value for the respective cycle is greater than or equal to a temperature threshold: wherein the predict is indicative of a number of the cycles for which the peak temperature value is greater than or equal to the temperature threshold being greater than or equal to a predefined number.

In some cases, the temperature threshold is between 39° C. and 41° C.

In some cases, the predefined number is two or more.

In some cases, the monitored record includes a monitored acceleration time series of monitored acceleration values over the given time period, the given time period including a plurality of identical and consecutive sub-periods, and each monitored acceleration value of the monitored acceleration values being indicative of an acceleration of the monitored animal over a respective sub-period of the sub-periods, wherein the processing circuitry is further configured to: determine the monitored acceleration values in the monitored acceleration time-series that are less than or equal to an acceleration threshold; and wherein the predict is also based on a determination that at least a predefined percentage of the monitored acceleration values are less than or equal to the acceleration threshold.

In some cases, the predefined percentage is all of the monitored acceleration values.

In some cases, the processing circuitry is further configured to: provide historical acceleration values for one or more animals, each historical acceleration value of the historical acceleration values being indicative of the acceleration of a respective animal of the one or more animals over a second respective sub-period, being earlier than and of an identical duration to the respective sub-period; and determine the acceleration threshold, based on the historical acceleration values.

In some cases, the monitored acceleration values are based on raw acceleration readings that are read by one or more acceleration sensors that are mounted on an animal tag assembly that is placed on the monitored animal.

In accordance with a second aspect of the presently disclosed subject matter, there is provided a method for predicting an illness, death or other abnormal condition of a monitored animal, the method comprising: providing a monitored record for the monitored animal, the monitored record including a monitored temperature time series of monitored temperature values that are indicative of a temperature of the monitored animal over a given time period: analyzing the monitored temperature time series: predicting the illness, death or other abnormal condition of the monitored animal within a given time duration of the given time period, based on the analysis, wherein the monitored temperature time series is a temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley; and notifying a user of the prediction.

In some cases, the monitored temperature time series is analyzed using a Machine Learning (ML) model, the ML model being trained based on a data repository of historical records for a plurality of animals, each historical record of the historical records including: (A) a historical temperature time series of historical temperature values that are indicative of the temperature of a respective animal of the plurality of animals over an earlier time period, being earlier than and of an identical duration to the given time period, and (B) a target field that indicates whether the respective animal became ill, died, or developed any other abnormal condition within the given time duration of the earlier time period.

In some cases, the ML model is a Switching Autoregressive Hidden Markov Model (SAR-HMM).

In some cases, for each cycle of the cycles, a temperature difference between a peak temperature value of the monitored temperature values in the respective cycle and a valley temperature value of the monitored temperature values in the respective cycle is greater than or equal to a predetermined difference.

In some cases, the predetermined difference is at least 4° C.

In some cases, the given time period is two to five days.

In some cases, the given time duration is two months or less.

In some cases, the monitored temperature values are based on raw temperature readings that are read by one or more temperature sensors that are mounted on an animal tag assembly that is placed on the monitored animal.

In some cases, the method further comprises: determining, for each cycle of the cycles, whether the peak temperature value for the respective cycle is greater than or equal to a temperature threshold: wherein the predicting is indicative of a number of the cycles for which the peak temperature value is greater than or equal to the temperature threshold being greater than or equal to a predefined number.

In some cases, the temperature threshold is between 39° C. and 41° C.

In some cases, the predefined number is two or more.

In some cases, the monitored record includes a monitored acceleration time series of monitored acceleration values over the given time period, the given time period including a plurality of identical and consecutive sub-periods, and each monitored acceleration value of the monitored acceleration values being indicative of an acceleration of the monitored animal over a respective sub-period of the sub-periods, wherein the method further comprises: determining the monitored acceleration values in the monitored acceleration time-series that are less than or equal to an acceleration threshold: wherein the predict is also based on a determination that at least a predefined percentage of the monitored acceleration values are less than or equal to the acceleration threshold.

In some cases, the predefined percentage is all of the monitored acceleration values.

In some cases, the method further comprises: providing historical acceleration values for one or more animals, each historical acceleration value of the historical acceleration values being indicative of the acceleration of a respective animal of the one or more animals over a second respective sub-period, being earlier than and of an identical duration to the respective sub-period; and determining the acceleration threshold, based on the historical acceleration values.

In some cases, the monitored acceleration values are based on raw acceleration readings that are read by one or more acceleration sensors that are mounted on an animal tag assembly that is placed on the monitored animal.

In accordance with a third aspect of the presently disclosed subject matter, there is provided a non-transitory computer readable storage medium having computer readable program code embodied therewith, the computer readable program code, executable by a processing circuitry of a computer to perform a method for predicting an illness, death or other abnormal condition of a monitored animal, the method comprising: providing a monitored record for the monitored animal, the monitored record including a monitored temperature time series of monitored temperature values that are indicative of a temperature of the monitored animal over a given time period: analyzing the monitored temperature time series: predicting the illness, death or other abnormal condition of the monitored animal within a given time duration of the given time period, based on the analysis, wherein the monitored temperature time series is a temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley; and notifying a user of the prediction.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a block diagram schematically illustrating one example of an operation of a prediction system for predicting an illness, death or other abnormal condition of a monitored animal, in accordance with the presently disclosed subject matter;

FIG. 2 is a block diagram schematically illustrating one example of a prediction system, in accordance with the presently disclosed subject matter:

FIG. 3 is a flowchart illustrating a first example of a sequence of operations for predicting an illness, death or other abnormal condition of a monitored animal, in accordance with the presently disclosed subject matter:

FIGS. 4A to 4C provide graphs of monitored temperature time series of monitored records, in accordance with the presently disclosed subject matter; and

FIG. 5 is a flowchart illustrating a second example of a sequence of operations for predicting an illness, death or other abnormal condition of a monitored animal, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “providing”, “analyzing”, “predicting”, “notifying”, “determining” or the like, include actions and/or processes, including, inter alia, actions and/or processes of a computer, that manipulate and/or transform data into other data, said data represented as physical quantities, e.g. such as electronic quantities, and/or said data representing the physical objects. The terms “computer”, “processor”, “processing circuitry” and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field-programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

As used herein, the phrase “for example.” “an additional example”, “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

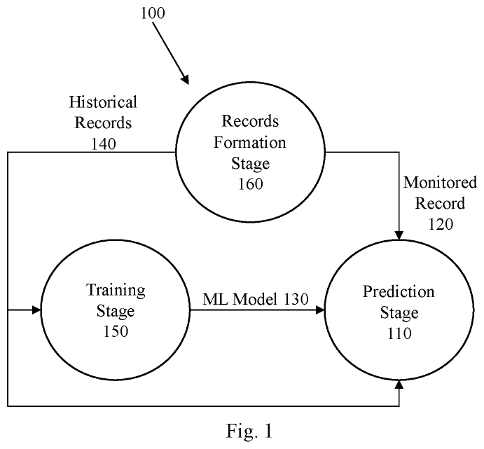

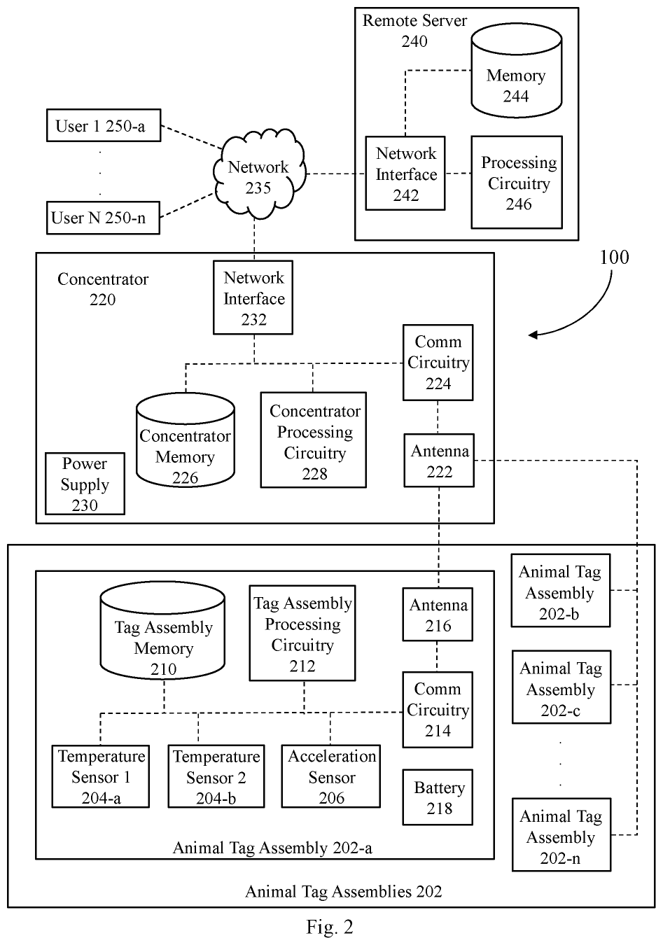

In embodiments of the presently disclosed subject matter, fewer, more and/or different stages than those shown in FIGS. 1, 3 and 5 may be executed. FIG. 2 illustrates a general schematic of the system architecture in accordance with embodiments of the presently disclosed subject matter. Each module in FIG. 2 can be made up of any combination of software, hardware and/or firmware that performs the functions as defined and explained herein. The modules in FIG. 2 may be centralized in one location or dispersed over more than one location. In other embodiments of the presently disclosed subject matter, the system may comprise fewer, more, and/or different modules than those shown in FIG. 2.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Attention is now drawn to FIG. 1, a block diagram schematically illustrating an operation of a prediction system 100 for predicting an illness, death or other abnormal condition of a monitored animal, in accordance with the presently disclosed subject matter.

In accordance with the presently disclosed subject matter, prediction system 100 is configured to include a prediction stage 110. Prediction system 100 is configured, in the prediction stage 110, to predict the illness, death or other abnormal condition of the monitored animal within a given time duration of a given time period by analyzing a monitored record 120 for the monitored animal that is associated with the given time period. In some cases, the monitored record 120 can include a monitored temperature time series of monitored temperature values that are indicative of a temperature of the monitored animal over the given time period, wherein the prediction of the illness, death or other abnormal condition of the monitored animal is based, at least in part, on an analysis of the monitored temperature time series. Additionally, or alternatively, in some cases, the monitored record 120 can include a monitored acceleration time series of monitored acceleration values that are indicative of an acceleration of the monitored animal over the given time period, wherein the prediction of the illness, death or other abnormal condition of the monitored animal is based, at least in part, on an analysis of the monitored acceleration time series.

In some cases, the given time period can be between two to five days (e.g., 48 hours, 60 hours, 72 hours, 84 hours, 96 hours, 108 hours, 120 hours, etc.). In some cases, the given time duration can be two months or less. In some cases, the given time duration can be one month or less.

In some cases, prediction system 100 can be configured to analyze the monitored record 120, using a Machine Learning (ML) model 130 that is trained based on a data repository of historical records 140 for a plurality of animals, as detailed below. In some cases, the ML model 130 can be trained by the prediction system 100, in a training stage 150 of the prediction system 100. In some cases, the ML model 130 can be a Switching Autoregressive Hidden Markov Model (SAR-HMM).

Prediction system 100 can be configured to include a records formation stage 160. Records formation stage 160 can be configured to provide the monitored record 120, as detailed further herein, inter alia with reference to FIG. 2. In some cases, records formation stage 160) can also be configured to provide the data repository of historical records 140, as detailed further herein, inter alia with reference to FIG. 2.

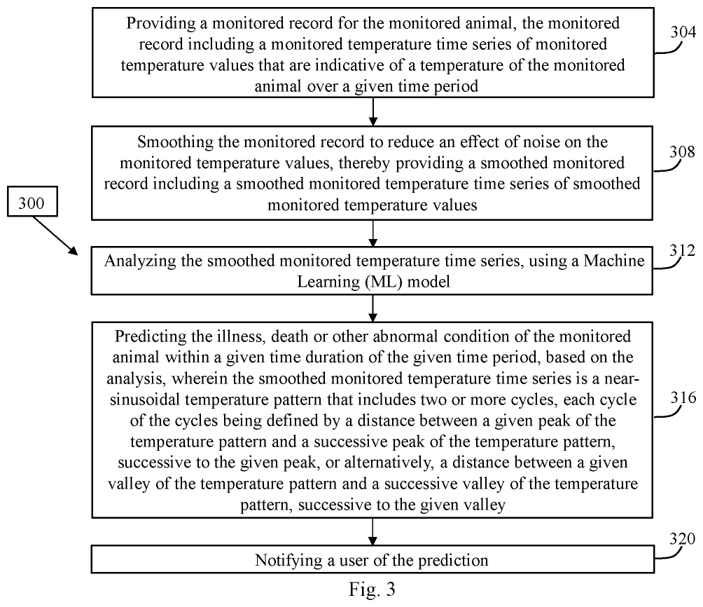

In some cases, each historical record of the historical records 140 can include: (A) a historical temperature time series of historical temperature values that are indicative of the temperature of a respective animal over an earlier time period, being earlier than and of an identical duration to the given time period, and (B) one or more target fields that indicate whether the respective animal became ill, died, or developed any other abnormal condition within the given time duration of the earlier time period. The ML model 130 can be trained, e.g. by training stage, based on at least selected historical records of the historical records 140) (possibly all of the historical records 140). Prediction system 100 can then be configured to analyze a monitored temperature time series of the monitored record 120 for the monitored animal, using the ML model 130, to predict the illness, death or other abnormal condition of the monitored animal within the given time duration of the given time period associated with the monitored record 120, as detailed further herein, inter alia with reference to FIG. 3.

In some cases, each historical record of the historical records 140 can include: (A) a historical acceleration time series of historical acceleration values that are indicative of the acceleration of a respective animal over an earlier time period, being earlier than and of an identical duration to the given time period, and (B) one or more target fields that indicate whether the respective animal became ill, died, or developed any other abnormal condition within the given time duration of the earlier time period.

In some cases, a ML model 130 can be trained, e.g. by training stage 150), based on at least selected records of the historical records 140. Prediction system 100 can then be configured to analyze a monitored acceleration time series of the monitored record 120 for the monitored animal, using the ML model 130, to predict the illness, death or other abnormal condition of the monitored animal within the given time duration of the given time period associated with the monitored record 120.

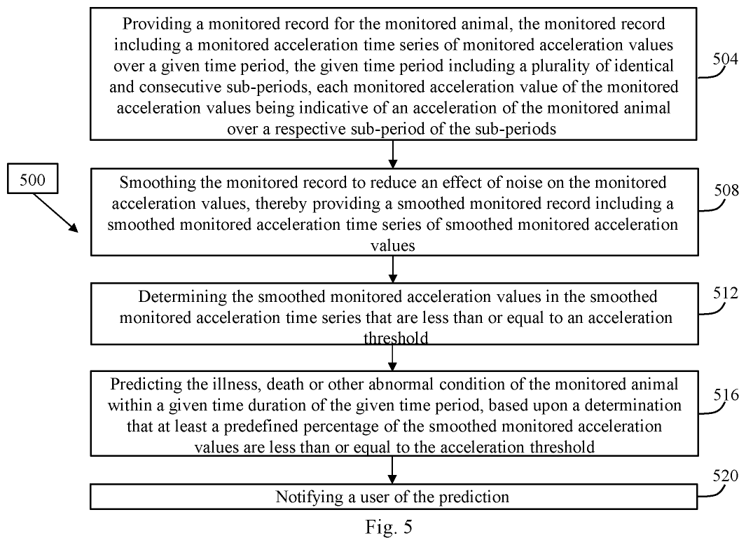

Alternatively, in some cases, a maximum acceleration threshold can be determined for monitored acceleration values of the monitored acceleration time series, based on relations between the historical acceleration time series of at least some of the historical records 140 and their corresponding target fields, as detailed further herein, inter alia with reference to FIG. 5. Prediction system 100 can then be configured to analyze the monitored acceleration values, in accordance with the maximum acceleration threshold, to predict the illness, death or other abnormal condition of the monitored animal within the given time duration of the given time period associated with the monitored record 120, as detailed further herein, inter alia with reference to FIG. 5.

In some cases, each historical record of the historical records 140 can include: (A) a historical temperature time series of historical temperature values that are indicative of the temperature of a respective animal over an earlier time period, being earlier than and of an identical duration to the given time period, (B) a historical acceleration time series of historical acceleration values that are indicative of the acceleration of the respective animal over the earlier time period, and (C) one or more target fields that indicate whether the respective animal became ill, died, or developed any other abnormal condition within the given time duration of the earlier time period. The ML model 130 can be trained, e.g. by training stage 150, based on at least selected records of the historical records 140).

In some cases, the ML model 130 can be a combined ML model that models both the historical temperature time series and the historical acceleration time series of at least selected historical records of the historical records 140. Prediction system 100 can be configured to predict the illness, death or other abnormal condition of the monitored animal within the given time duration of the given time period associated with the monitored record 120 by analyzing the monitored temperature time series and the monitored acceleration time series of the monitored record 120, using the combined ML model 130).

Alternatively, in some cases, the ML model 130 can model the historical temperature time series of the selected historical records 140 and not the historical acceleration time series of the selected historical records 140. Prediction system 100 can be configured to provide a temperature-based prediction of illness, death or other abnormal condition of the monitored animal within the given time duration of the given time period associated with the monitored record 120 by analyzing the monitored temperature time series of the monitored record 120, using the ML model 130, as detailed further herein, inter alia with reference to FIG. 3. Moreover, a maximum acceleration threshold can be determined for monitored acceleration values of the monitored acceleration time series of the monitored record 120, based on relations between the historical acceleration time series of at least some of the historical records 140 and their corresponding target fields, as detailed further herein, inter alia with reference to FIG. 5. Prediction system 100 can be configured to analyze the monitored acceleration values, in accordance with the maximum acceleration threshold, to provide an acceleration-based prediction of the illness, death or other abnormal condition of the monitored animal within the given time duration of the given time period associated with the monitored record 120, as detailed further herein, inter alia with reference to FIG. 5. Prediction system 100 can be configured, based on the temperature-based prediction and the acceleration-based prediction, to predict (e.g., provide a final prediction of) the illness, death or other abnormal condition of the monitored animal within the given time duration of the given time period associated with the monitored record 120.

Attention is now drawn to FIG. 2, a block diagram schematically illustrating one example of a prediction system 100, in accordance with the presently disclosed subject matter. In accordance with the presently disclosed subject matter, prediction system 100 can be configured, in some cases, to provide, in records formation stage 160, a data repository of historical records 140 for a plurality of animals. Each historical record of the historical records 140 can include at least one of: (a) a historical temperature time series of historical temperature values that are indicative of a temperature of a respective animal of the animals over an earlier time period, as detailed earlier herein, inter alia with reference to FIG. 1, or (b) a historical acceleration time series of historical acceleration values that are indicative of an acceleration of the respective animal over the earlier time period, as detailed earlier herein, inter alia with reference to FIG. 1. Each historical record of the historical records 140 also includes one or more target fields that indicate whether the respective animal associated with the respective historical record became ill, died, or developed any other abnormal condition within the given time duration of the earlier time period.

In some cases, one or more of the historical records 140 can be obtained by the prediction system 100 from an external system, external to the prediction system 100.

System 100 can also be configured to provide, in records formation stage 160, one or more monitored records 120 for one or more monitored animals. Each monitored record of the monitored records 120 can include at least one of: (a) a monitored temperature time series of monitored temperature values that are indicative of a temperature of a respective monitored animal of the monitored animals over a given time period, or (b) a monitored acceleration time series of monitored acceleration values that are indicative of an acceleration of the respective monitored animal over the given time period.

The temperature values of a temperature time series that is associated with a respective animal, whether historical temperature values or monitored temperature values, are based on raw temperature readings obtained from the respective animal by one or more temperature sensors (e.g., 204-a, 204-b). Likewise, the acceleration values of an acceleration time series that is associated with a respective animal, whether historical acceleration values or monitored acceleration values, are based on raw acceleration readings obtained from the respective animal by one or more acceleration sensors (e.g., 206).

In some cases, as illustrated in FIG. 2, the temperature sensors (e.g., 204-a, 204-b) and the acceleration sensors (e.g., 206) for providing raw temperature readings and raw acceleration readings, respectively, for a respective animal are mounted on an animal tag assembly (e.g., 202-a) that is placed on the respective animal. As illustrated in FIG. 2, system 200 can be configured to include one or more animal tag assemblies 202 (e.g., 202-a, 202-b, 202-c, . . . 202-n) that are placed on one or more animals. The animals on which the animal tag assemblies 202 are placed are referred to hereinafter, interchangeably, as “tagged animals”. Each tagged animal can have one or more animal tag assemblies 202 placed thereon. At least one of the animal tag assemblies 202 that is placed on a respective tagged animal can be configured to monitor at least one of a temperature or an acceleration of the respective tagged animal. For example, animal tag assembly 202-a is configured to monitor both a temperature and an acceleration of the tagged animal on which the animal tag assembly 202-a is placed. Moreover, in some cases, at least one of the animal tag assemblies 202 that is placed on a respective tagged animal can be configured to enable the respective tagged animal to be uniquely identified. In some cases, the unique identification of the respective tagged animal can be achieved by including on an animal tag assembly (e.g., 202-a. 202-b, 202-c, . . . , 202-n) that is placed on the respective tagged animal one or more of: a digital token, one or more RFID devices, one or more electronic chips or sensors, a printed label, a name tag, or any combination thereof for uniquely identifying the respective tagged animal. In some cases, a single animal tag assembly (e.g., 202-a, 202-b, 202-c . . . , 202-n) that is placed on a respective tagged animal can be configured to both uniquely identify the respective tagged animal and to monitor at least one of a temperature or an acceleration of the respective tagged animal.

In some cases, a tagged animal can be uniquely identified by a separate identification device(s) that is not included in any of the one or more animal tag assemblies 202 that are placed on the tagged animal. The separate identification device(s) can, in some cases, be affixed to the tagged animal, placed on the tagged animal, or implanted under the skin of the tagged animal. For example, a RFID device can be implanted under the skin of the tagged animal for uniquely identifying the tagged animal. In some cases, the separate identification device(s) can include one or more of: a digital token, one or more RFID devices, one or more electronic chips or sensors, a printed label, a name tag, or any combination thereof for uniquely identifying the tagged animal.

In some cases, in which one or more RFID devices are used for identifying a tagged animal (whether the RFID devices are included on an animal tag assembly 202 or separate from an animal tag assembly 202), the tagged animal can be identified by a RFID reader (e.g., in concentrator 120) that reads the data that is stored in the RFID devices for identifying the tagged animal. In some cases, at least one of the RFID devices can include a low frequency passive RFID device. Additionally, or alternatively, in some cases, at least one of the RFID devices can include an active RFID device.

The animal tag assemblies 202 that are placed on a respective tagged animal can be placed on one or more of: an ear, a neck, an ankle (e.g., ankle bracelet), a tail, a rectum, a vagina, an eye, a nose, or any other location on the respective tagged animal.

In some cases, at least one of the animal tag assemblies 202 that is placed on a respective tagged animal (e.g., animal tag assembly 202-a) can be configured to include one or more temperature sensors (e.g., 204-a, 204-b). The temperature sensors (e.g., 204-a, 204-b) can be configured to provide raw temperature readings that are indicative of a temperature of the tagged animal. The temperature sensors (e.g., 204-a, 204-b), whether or not included on/within an animal tag assembly (e.g., 202-a), can include, for example, at least one of: one or more infrared (IR) temperature sensors, one or more thermocouples, one or more thermistors or one or more thermopile detectors. The temperature sensors (e.g., 204-a, 204-b) that are included in a respective animal tag assembly (e.g., 202-a) can be positioned on/within the respective animal tag assembly (e.g., 202-a) in close proximity to or in direct contact with the tagged animal that is associated with the respective animal tag assembly (e.g., 202-a) to provide raw temperature readings that more accurately indicate the temperature of the tagged animal.

In some cases, an animal tag assembly (e.g., 202-a) that includes one or more temperature sensors (e.g., 204-a. 204-b) can be disposed on/within the ear of a tagged animal. The temperature sensors (e.g., 204-a, 204-b) can be configured to provide one or more raw ear temperature readings for the tagged animal at any given time, and, in some cases, one or more ambient temperature readings of an ambient temperature in the vicinity of the animal tag assembly (e.g., 202-a) at the given time. The raw ear temperature readings and the ambient temperature readings that are provided by the temperature sensors (e.g., 204-a, 204-b) can include, for example, one or more of: temperature readings of a temperature of a portion of the inner ear (i.e., an Inner Ear Temperature (IET)) of the tagged animal: temperature readings of an ambient temperature of the ear canal (i.e., an Ambient Temperature Near Canal (ANC)) of the tagged animal: temperature readings of a temperature of a portion of the ear surface/outer ear (i.e., an Ear Surface Temperature (EST)) of the tagged animal: or temperature readings of an ambient temperature near a printed circuit board (PCB) on the animal tag assembly (e.g., 202-a) (i.e., an Ambient Temperature near PCB Surface (APCB)). The temperature readings of the ambient temperatures at/within the ear of the tagged animal (e.g., the ANC and/or the APCB) can be used to provide adjusted temperature readings, and thereby compensate for the effects of ambient temperature on the raw ear temperature readings (e.g., the IET and/or the EST) for the tagged animal.

By way of example, the adjusted temperature reading at any time ‘t’ can be calculated as follows:

$$Adj . Temp ( t ) = A \times IET ( t ) + B \times EST ( t ) + C \times ( ANC ( t ) + APCB ( t ) ) \qquad ( Equation 1 )$$

where A, B, and C are weighting constants.

In some cases, the value of weighting constant A can be greater than the value of weighting constant B. In some cases, the values of one or more of the weighting constants A, B, or C can be acquired from a calibration table.

It is to be noted herein that, in some cases, raw temperature readings (for example, raw ear temperature readings) can be adjusted to compensate for one or more external conditions (e.g., external environmental conditions) that may impact the raw temperature readings, including, but not limited to, local (e.g., ambient) temperatures, local humidity and/or local atmospheric pressure, thereby resulting in adjusted temperature readings. For the purposes of this disclosure, the adjusted temperature readings are considered to be raw temperature readings.

It is to be noted that the temperature sensors (e.g., 204-a, 204-b) can be configured to provide more than one raw temperature reading of an animal at any given time, whether or not the temperature sensors (e.g., 204-a, 204-b) are mounted on an animal tag assembly 202.

Attention is now redirected to the embodiment in which one or more temperature sensors (e.g., 204-a. 204-b) are disposed on/within an ear of a tagged animal. In some cases, one of the temperature sensors (e.g., 204-a, 204-b) can be configured to provide two different temperature readings. For example, one of the temperature sensors (e.g., 204-a, 204-b) can be configured to provide temperature readings of an IET and an ANC of the tagged animal. As an additional example, one of the temperature sensors (e.g., 204-a, 204-b) can be configured to provide temperature readings of an EST and an APCB of the tagged animal.

In some cases, at least one of the temperature sensors (e.g., 204-a, 204-b) that are disposed on/within the ear of the tagged animal can be configured to provide temperature readings of a differential temperature between a proximate part of the ear (e.g., ear canal, outer ear) of the tagged animal and the ambient environment to compensate for the effects of ambient temperature on the ear temperature (IET and/or EST) of the tagged animal.

In some cases, an animal tag assembly (e.g., 202-a) that includes one or more temperature sensors (e.g., 204-a. 204-b) can be placed at another location on the skin of a tagged animal, other than the ear of the tagged animal. For example, the animal tag assembly (e.g., 202-a) can be placed on the rectum, vagina, eyes, or nose of the tagged animal.

In some cases, at least one of the animal tag assemblies 202 that is placed on a respective tagged animal (e.g., animal tag assembly 202-a) can be configured to include one or more acceleration sensors (e.g., 206). The acceleration sensors (e.g., 206) can be configured to provide raw acceleration readings that are indicative of an acceleration of the tagged animal.

In some cases, at least one of the acceleration sensors (e.g., 206) that is included in a respective animal tag assembly (e.g., 202-a) can be an accelerometer, for example a three-axis accelerometer that is configured to measure acceleration of the tagged animal that is associated with the respective animal tag assembly (e.g., 202-a) in three axes. Additionally, or alternatively, in some cases, at least one of the acceleration sensors (e.g., 206) can be part of a consolidated Inertial Management Unit (IMU) that also includes a gyroscope and/or magnetometer.

In some cases, an animal tag assembly (e.g., 202-a) that includes one or more acceleration sensors (e.g., 206) can be disposed on/within the ear, the neck, the ankle (e.g., via ankle bracelets), the tail or any other relevant location on the skin of the tagged animal that is associated with the animal tag assembly (e.g., 202-a) for measuring acceleration of the tagged animal.

Each of the animal tag assemblies 202 can be configured to include a tag assembly memory 210 (e.g. a database, a storage system, a memory including Read Only Memory-ROM (e.g., Electrically Erasable Programmable ROM (EEPROM)), Random Access Memory-RAM, or any other type of memory, etc.) configured to store data. The stored data can include, for example, the raw temperature readings and/or the raw acceleration readings for the tagged animal. In some cases, tag assembly memory 210 can be further configured to enable retrieval and/or update and/or deletion of the stored data. It is to be noted that in some cases, tag assembly memory 210 can be distributed.

Each of the animal tag assemblies 202 can be further configured to include a tag assembly processing circuitry 212. Tag assembly processing circuitry 212 can be configured to include one or more tag assembly processing units, being, for example, central processing units, microprocessors, microcontrollers (e.g. microcontroller units (MCUs)) or any other computing devices or modules, including multiple and/or parallel and/or distributed processing units, which are adapted to independently or cooperatively process data for controlling animal tag assembly 202 resources and for enabling operations related to animal tag assembly 202 resources.

Tag assembly processing circuitry 212 of a respective animal tag assembly (e.g., 202-a) can be configured to obtain the raw temperature readings from the one or more temperature sensors (e.g., 204-a, 204-b) on/within the animal tag assembly (e.g., 202-a). In some cases, two or more raw temperature readings can be simultaneously obtained by the tag assembly processing circuitry 212 (i.e., can be simultaneously provided by the temperature sensors (e.g., 204-a, 204-b)). In some cases, the temperature sensors (e.g., 204-a, 204-b) can be configured to provide the raw temperature readings, for example to the tag assembly processing circuitry 212, at predetermined intervals (e.g., every minute, every five minutes, every 10 minutes, every 15 minutes, every 30 minutes, every hour, etc.).

Tag assembly processing circuitry 212 of a respective animal tag assembly (e.g., 202-a) can also be configured to obtain the raw acceleration readings from the one or more acceleration sensors (e.g., 206) on/within the animal tag assembly (e.g., 202-a). In some cases, two or more raw acceleration readings can be simultaneously obtained by the tag assembly processing circuitry 212 (i.e., can be simultaneously provided by the one or more acceleration sensors (e.g., 206)). In some cases, the one or more acceleration sensors (e.g., 206) can be configured to provide the raw acceleration readings, for example to the tag assembly processing circuitry 212, at predetermined intervals (e.g., every twentieth of a second, every tenth of a second, every half a second, every second, every two seconds, etc.).

In some cases, one or more (e.g., all) of the animal tag assemblies 202 (e.g., animal tag assembly 202-a) can be configured to include communications circuitry 214 and an antenna 216. In some cases, as illustrated in FIG. 2, communications circuitry 214 and antenna 216 can be configured to transmit data, for example, to a concentrator 220, and to receive data or instructions, for example, from the concentrator 220. Communications circuitry 214 and antenna 216 can be configured to operate in any frequency band known in the art. In some cases, the communications circuitry 214 and the antenna 216 can be configured to operate in a Radio Frequency (RF) band. In some cases, the communications circuitry 214 and the antenna 216 can be configured to operate in a selected band (e.g., a band between 902 MHz and 928 MHz). It is noted herein that the antenna 216 can be of any type known in the art, including, but not limited to, an embedded antenna or an external antenna.

In some cases, one or more (e.g., all) of the animal tag assemblies 202 (e.g., animal tag assembly 202-a) can be communicatively coupled to a concentrator 220 via a local communications link, for example, a local wireless communications link. For example, the communications circuitry 214 and antenna 216 of a respective animal tag assembly (e.g., 202-a) can be configured to wirelessly communicate with a concentrator 220, for example, via the antenna 222 and the communications circuitry 224 of concentrator 220. In some cases, the communications circuitry 214 of the respective animal tag assembly (e.g., 202-a) can be configured to include a radio frequency (RF) module suitable for transmitting one or more RF signals to the communications circuitry 224 of the concentrator 220. The communications circuitry 214 of a respective animal tag assembly (e.g., 202-a) and the communications circuitry 224 of concentrator 220 that communicates with the respective animal tag assembly (e.g., 202-a) can be compatible with any wireless protocol known in the art, such as, but not limited to, Bluetooth, Low Energy Bluetooth, WiFi, RFID, and the like. A respective animal tag assembly (e.g., 202-a) can be configured to transmit raw readings (e.g., raw temperature readings and/or raw acceleration readings) that are read by the sensors (e.g., 204-a, 204-b, 206) in the respective animal tag assembly (e.g., 202-a) and/or data values (e.g., temperature values and/or acceleration values) that are derived from the raw readings to a concentrator 220. In some cases, the raw readings and/or the data values can be transmitted from the respective animal tag assembly (e.g., 202-a) to the concentrator 220, upon the respective animal tag assembly (e.g., 202-a) being interrogated by the concentrator 220.

In some cases (not as illustrated in FIG. 2), a respective animal tag assembly (e.g., 202-a) can be communicatively coupled to a communications network 235, being any wireless and/or wireline network protocol known in the art. The respective animal tag assembly (e.g., 202-a) can be communicatively coupled to the communications network 235, via a network interface (not shown in FIG. 2), to directly communicate with other computerized devices, including, inter alia, a remote server 240). In some cases, the communications network 235 can include, but is not limited to, an Internet or an Intranet (e.g., LAN, WLAN or the like). In some cases, communications network 235 can include a cloud-based architecture.

Each of the animal tag assemblies 202 can be configured to include a power supply (e.g., battery 218) to power the electronic components that are mounted on the respective animal tag assembly (e.g., 202-a, 202-b, 202-c . . . , 202-n). The power supply can be, for example, one or more batteries (e.g., battery 218), one or more power generating devices (e.g., piezoelectric devices, photovoltaic cells or the like), a combination of one or more batteries and power generating devices, or the like. It is noted herein that each of the animal tag assemblies 202 can utilize any battery technology known in the art.

In some cases, a concentrator 220) (or a network of concentrators) can be configured to communicate with the animal tag assemblies 202, e.g. via antenna 122 and communications circuitry 124, to acquire data from the animal tag assemblies 202.

Concentrator 220 can be configured to include a concentrator memory 226 (e.g., a database, a storage system, a memory including Read Only Memory-ROM (e.g., Electrically Erasable Programmable ROM (EEPROM)), Random Access Memory-RAM, or any other type of memory, etc.) configured to store data. The stored data can include, for example, at least one of: raw temperature readings and/or raw acceleration readings, historical temperature values and/or historical acceleration values of historical records 140, or monitored temperature values and/or monitored acceleration values of monitored records 120 for animals that are associated with the animal tag assemblies 202 that are coupled to the concentrator 220. In some cases, concentrator memory 226 can be further configured to enable retrieval and/or update and/or deletion of the stored data. It is to be noted that in some cases, concentrator memory 226 can be distributed.

Concentrator 220) can be further configured to comprise a concentrator processing circuitry 228 including one or more concentrator processing units, being, for example, central processing units, microprocessors, microcontrollers (e.g. microcontroller units (MCUs)) or any other computing devices or modules, including multiple and/or parallel and/or distributed processing units, which are adapted to independently or cooperatively process data for controlling concentrator 220 resources and for enabling operations related to concentrator 220 resources. In some cases, as detailed further herein, concentrator processing circuitry 228 can be configured to generate at least one of: (a) historical temperature values and/or historical acceleration values of historical records 140 or (b) monitored temperature values and/or monitored acceleration values of monitored records 120, based on raw temperature readings and/or raw acceleration readings from one or more animals.

Concentrator 220 can be configured to include a power supply 230. Power supply 230 can be any power supply that is known in the art including, but not limited to, a battery or a transformer configured to convert AC power to DC power.

Concentrator 220 can be configured to include a network interface 232, for example, to communicate with remote server 240 via communications network 235. Network interface 232 can be configured to communicate, for example with the remote server 240), using any network protocol known in the art including, but not limited to, Ethernet, WiFi, 3G, 4G, 4G LTE, 5G, or the like. Furthermore, the concentrator 220) and remote server 240 can be communicatively coupled via any wireless or wireline mechanism known in the art. It is further noted that multiple network protocols may be utilized by network interface 232 for communications. In some cases, the concentrator 220) can include multiple network interfaces. In some cases, the communications network 235 can include, but is not limited to, an Internet or an Intranet (e.g., LAN, WLAN or the like). In some cases, communications network 235 can include a cloud-based architecture.

Remote server 240) can include one or more servers. In some cases, at least one of the servers can be a cloud-based server. In some cases, the remote server 240 can be coupled to one or more concentrators 220, e.g., via communications network 235, as illustrated in FIG. 2. Additionally, or alternatively, in some cases, the remote server 240 can be coupled to the animal tag assemblies (e.g., 202), e.g., via communications network 235.

Remote server 240) can be configured to include a network interface 242. Network interface 242 can be configured to communicate, via communications network 235, using any network protocol known in the art including, but not limited to, Ethernet, WiFi, 3G, 4G, 4G LTE, 5G, or the like. In some cases, network interface 242 can be configured to communicate, e.g., via communications network 235, with one or more concentrators 220, as illustrated in FIG. 2. Additionally, or alternatively, in some cases, network interface 242 can be configured to communicate, e.g., via communications network 235, with the animal tag assemblies (e.g., 202).

Remote server 240) can be configured to include a server memory 244 (e.g., a database, a storage system, a memory including Read Only Memory-ROM (e.g., Electrically Erasable Programmable ROM (EEPROM)), Random Access Memory-RAM, or any other type of memory, etc.) configured to store data. The stored data can include, for example, at least one of: raw temperature readings and/or raw acceleration readings, historical temperature values and/or historical acceleration values of the historical records 140, or monitored temperature values and/or monitored acceleration values of the monitored records 120 for animals (e.g., animals that are associated with animal tag assemblies 202). In some cases, server memory 244 can be further configured to enable retrieval and/or update and/or deletion of the stored data. It is to be noted that in some cases, server memory 244 can be distributed.

Remote server 240) can be further configured to comprise a server processing circuitry 246. Server processing circuitry 246 can be configured to include one or more server processing units, for example, central processing units, microprocessors, microcontrollers (e.g. microcontroller units (MCUs)) or any other computing devices or modules, including multiple and/or parallel and/or distributed processing units, which are adapted to independently or cooperatively process data for controlling remote server 240 resources and for enabling operations related to remote server 240 resources. In some cases, server processing circuitry 246 can be configured to generate at least one of: (a) historical temperature values and/or historical acceleration values of historical records 140 or (b) monitored temperature values and/or monitored acceleration values of monitored records 120, based on raw temperature readings and/or raw acceleration readings from one or more animals. In some cases, the generation of at least one of the: (a) historical temperature values and/or historical acceleration values or (b) monitored temperature values and/or monitored acceleration values can be performed by other processing circuitries (e.g., 212, 228) in the system 100.

In some cases, the historical temperature values and the monitored temperature values can be raw temperature readings. In some cases, each raw temperature reading can be a temperature value (e.g., a historical temperature value or a monitored temperature value) of a respective record (e.g., a historical record 140 or a monitored record 120). Alternatively, in some cases, each temperature value for a respective animal can be one of a plurality of raw temperature readings that are read for the respective animal. For example, the raw temperature readings for the respective animal can be read at first predetermined intervals (e.g., every 15 minutes), and the historical or monitored temperature values for a respective record associated with the respective animal can be generated at second predetermined intervals (e.g., every hour), the second predetermined intervals being greater than the first predetermined intervals. In the example in which the raw temperature readings are read every 15 minutes, and the temperature values are generated every hour, a single temperature value for a given hour can be selected, in some cases, to be one of the four raw temperature readings read over the given hour. As an alternative example, the raw temperature readings can be read at the same intervals that the historical and monitored temperature values are generated, e.g., hourly. However, multiple raw temperature readings can be read simultaneously, and a respective temperature value can be selected to be one of the multiple raw temperature readings.

In some cases, each of the historical temperature values and the monitored temperature values can be generated by processing a plurality of raw temperature readings. For example, the raw temperature readings for a respective animal can be read at predetermined intervals, for example, every 15 minutes. In some cases, a plurality of raw temperature readings (e.g., three or four readings) can be simultaneously read at the predetermined intervals. If, for example, each temperature value of the temperature values that is part of a temperature time series of a record (e.g., historical record 140 or monitored record 120) that is associated with the respective animal is provided on an hourly basis over the given time period of the record, and a single raw temperature reading is read every 15 minutes, each temperature value can be an average of the four temperature readings that are read over the course of the hour, or can be generated using any mathematical operation that is performed on the four temperature readings or a subset thereof. In some cases, if a plurality of raw temperature readings are simultaneously read at predetermined intervals, e.g. three raw temperature readings are simultaneously read every 15 minutes, a single raw temperature reading can be selected from the plurality of raw temperature readings that are simultaneously read for further processing. Alternatively, an average of the raw temperature readings that are simultaneously read can be calculated or any other mathematical operation can be performed on the raw temperature readings that are simultaneously read, wherein the results of the average or the other mathematical operation can be used to generate a respective temperature value of a record. For example, three raw temperature readings for a respective animal can be read every 15 minutes. An average of the three raw temperature readings that are read at a given time can be calculated. As a result, at the end of one hour, four averages of three temperature readings can be provided, based on which a respective temperature value of a record can be generated. For example, one of the four averages can be selected to be the respective temperature value. As an additional example, a mathematical operation, e.g., the taking of an average, can be performed on the four averages, to provide the respective temperature value. It is to be noted that any method by which a respective temperature value of a record (e.g., monitored record 120 or historical record 140) is generated based on raw temperature readings is covered by the present disclosure.

Historical acceleration values of historical records 140 and monitored acceleration values of monitored records 120 can be generated as follows. A plurality of raw acceleration readings can be read for each historical acceleration value or monitored acceleration value that is generated. In some cases, the raw acceleration readings can be provided at predetermined intervals (e.g., every twentieth of a second, every tenth of a second, every half a second, every second, every two seconds, etc.), as discussed above. Each historical acceleration value or monitored acceleration value can be calculated, using any mathematical formula or algorithm, based on a plurality of acceleration readings that are read over a sub-period (e.g., one hour) of the given time period (e.g., two to five days) that is associated with a respective record (e.g., historical record 140, monitored record 120). In some cases, each historical acceleration value or monitored acceleration value that is associated with a respective sub-period (e.g., one hour) can be determined by calculating a total acceleration of the acceleration readings over the course of the respective sub-period, as detailed below.

An example of a calculation of a historical acceleration value or a monitored acceleration value, based on raw acceleration readings that are read by a three-axis accelerometer (e.g., 206) on/within an animal tag assembly (e.g., 202-a), is now provided. In this example, the animal tag assembly (e.g., 202-a) is located on an ear of a tagged animal. First acceleration readings (e.g., aₓ) that are read by the accelerometer (e.g., 206) represent acceleration along an X-axis, and are indicative of leftward and rightward movements of the tagged animal's head. Second acceleration readings (e.g., a_{y}) that are read by the accelerometer (e.g., 206) represent acceleration along a Y-axis, and are indicative of forward and backward movements of the tagged animal's head. Third acceleration readings (e.g., a_{z}) that are read by the accelerometer (e.g., 206) represent acceleration along a Z-axis, and are indicative of up and down movements of the tagged animal's head. It is to be noted herein that the animal tag assembly (e.g., 202-a) on/within which the accelerometer (e.g., 206) is placed can be located on any part of the tagged animal's body. As a corollary thereto, the acceleration readings can be indicative of other movements by the tagged animal, other than movements of the tagged animal's head. In the above example, the acceleration that is measured by the three-axis accelerometer (e.g., 206) is defined in a Cartesian coordinate system including Cartesian coordinates (aₓ, a_{y}, a_{z}). However, the acceleration that is measured by the three-axis accelerometer (e.g., 106) can be defined in a different coordinate system, for example a spherical coordinate system including spherical coordinates (aᵣ, a_{θ}, a_{φ}).

Returning to the above example of the three-axis accelerometer (e.g., 206), each historical acceleration value or monitored acceleration value for a respective sub-period can be calculated based on raw acceleration readings. For example, in some cases, each historical acceleration value or monitored acceleration value can be calculated as follows. The absolute values of the first acceleration readings (e.g., aₓ), representing acceleration along an X-axis, over a respective sub-period (e.g., an hour) can be added, resulting in a cumulative first acceleration for the respective sub-period. Moreover, the absolute values of the second acceleration readings (e.g., a_{y}), representing acceleration along a Y-axis, over the respective sub-period can be added, resulting in a cumulative second acceleration for the respective sub-period. In addition, the absolute values of the third acceleration readings (e.g., a_{z}), representing acceleration along a Z-axis, over the respective sub-period can be added, resulting in a cumulative third acceleration for the respective sub-period.

A specific example of the calculation of the cumulative first acceleration, the cumulative second acceleration and the cumulative third acceleration for the respective sub-period is now provided. In this example, the cumulative first acceleration, the cumulative second acceleration and the cumulative third acceleration are based on acceleration readings that are read by the three-axis accelerometer (e.g., 206) every second over the period of an hour. That is, the cumulative first acceleration is based on 3600 first acceleration readings (e.g., aₓ), the cumulative second acceleration is based on 3600 second acceleration readings (e.g., aₓ), and the cumulative third acceleration is based on 3600 third acceleration readings (e.g., a_{z}). In this case, the cumulative first acceleration is represented by the equation: Σaₓ=|aₓ₁|+|aₓ₂|+|aₓ₃|+ . . . +| . . . +aₓ₃₆₀₀|. The cumulative second acceleration is represented by the equation: Σ_{ay}=|a_{y1}|+|a_{y2}|+|a_{y3}|+ . . . +a_{y36}|. The cumulative third acceleration is represented by the equation: Σa_{z}=|a_{z1}|+|a_{z2}|+|a_{z3}|+ . . . +|a_{z3600}|.

In some cases, the calculation of a historical acceleration value or monitored acceleration value for a respective sub-period for a tagged animal, based on the cumulative first acceleration, the cumulative second acceleration, and the cumulative third acceleration for the respective sub-period, can represent the total acceleration of the tagged animal. For example, the historical acceleration value or the monitored acceleration value for the respective sub-period can be calculated as follows:

$$Acceleration Value = \sqrt{{( Σ {a}_{x} )}^{2} + {( Σ {a}_{y} )}^{2} + {( Σ {a}_{z} )}^{2}} \qquad ( Equation 2 )$$

As an additional example, the historical acceleration value or the monitored acceleration value can be calculated as follows:

$$Acceleration Value = \vert Σ ax \vert + \vert Σ ay \vert + \vert Σ az \vert \qquad ( Equation 3 )$$

It is to be noted that, in some cases, the historical acceleration value or the monitored acceleration value for the respective sub-period can be calculated, based on the cumulative first acceleration, the cumulative second acceleration, and the cumulative third acceleration for the respective sub-period, using a different algorithm or formula than provided above. It is to be further noted that in the case that the acceleration readings are read by a three-axis accelerometer (e.g., 206), the first acceleration readings (e.g., aₓ), the second acceleration readings (e.g., a_{y}) and the third acceleration readings (e.g., a_{z}) can be processed in a different manner than provided above, i.e., not processed to provide a cumulative first acceleration, a cumulative second acceleration and a cumulative third acceleration, as provided above.

In some cases, a historical acceleration value or a monitored acceleration value for the respective sub-period can be calculated based on raw acceleration readings that are read by a single axis accelerometer or a two-axis accelerometer. For example, in the case of a single axis accelerometer (e.g., 206), the absolute values of the acceleration readings (e.g., aₓ) of the acceleration of the tagged animal along the axis of the accelerometer (e.g., 106) over the respective sub-period can be added, resulting in a historical acceleration value or a monitored acceleration value for the respective sub-period, as follows:

$$Accleration Value = \vert {a}_{x} \vert \qquad ( Equation 4 )$$

As a further example, in the case of a two-axis accelerometer (e.g., 206) that measures acceleration along an X-axis and a Y-axis, a historical acceleration value or a monitored acceleration value for a respective sub-period can be calculated based on a cumulative first acceleration and a cumulative second acceleration. The cumulative first acceleration can be calculated, for example, by adding the absolute values of first acceleration readings (e.g., aₓ), representing acceleration along the X-axis, over the respective sub-period. Moreover, the cumulative second acceleration can be calculated, for example, by adding the absolute values of second acceleration readings (e.g., a_{y}), representing acceleration along the Y-axis, over the respective sub-period. In some cases, the historical acceleration value or monitored acceleration value can be calculated as follows:

$$Acceleration Value = \sqrt{{( Σ {a}_{x} )}^{2} + {( Σ {a}_{y} )}^{2}} \qquad ( Equation 5 )$$

In some cases, the historical acceleration value or monitored acceleration value can be calculated as follows:

$$Acceleration Value = \vert Σ ax \vert + \vert Σ ay \vert \qquad ( Equation 6 )$$

It is to be noted that, in some cases, in the case of a two-axis accelerometer (e.g., 206), the historical acceleration value or monitored acceleration value for a respective sub-period can be calculated based on the cumulative first acceleration and the cumulative second acceleration for the respective sub-period using a different algorithm or formula than provided above. It is to be further noted that in the case that the acceleration readings are read by a two-axis accelerometer (e.g., 206), the first acceleration readings (e.g., aₓ) and the second acceleration readings (e.g., a_{y}) can be processed in a different manner than provided above, i.e., not processed to provide a cumulative first acceleration and a cumulative second acceleration as provided above.

System 100 can be configured, e.g. using server processing circuitry 246, to provide one or more monitored records 120 based on at least one of: one or more monitored temperature time series of monitored temperature values or one or more monitored acceleration time series of monitored acceleration values. Moreover, in some cases, system 100 can be configured, e.g. using server processing circuitry 246, to provide a plurality of historical records 120 based on at least one of: a plurality of historical temperature time series of historical temperature values or a plurality of historical acceleration time series of historical acceleration values. In some cases, the plurality of historical records 120 can be smoothed historical records 120 that are smoothed, e.g., using a Kalman filter, to reduce an effect of random noise on the historical temperature values and the historical acceleration values.

One or more user devices (e.g., 250)-a . . . , 250)-n) can be connected to the prediction system 100. The one or more user devices (e.g., 250-a, . . . , 250)-n) can include, but are not limited to, one or more desktop computers, one or more tablet computers, one or more mobile phones (e.g., smartphones), one or more wearable devices (e.g., smartwatches) or the like.

The one or more user devices (e.g., 250)-a, . . . , 250)-n) can be communicatively coupled, directly or indirectly, to the remote server 240 (e.g., via the communications network 235).

In some cases, one or more user devices (e.g., 250)-a, . . . , 250)-n) can be communicatively coupled, directly or indirectly, to one or more concentrators 220, e.g. via communications network 235.

Attention is now drawn to FIG. 3, a flowchart illustrating a first example of a sequence of operations 300 for predicting an illness, death or other abnormal condition of a monitored animal, in accordance with the presently disclosed subject matter.

In accordance with the presently disclosed subject matter, prediction system 100 can be configured to predict an illness, death or other abnormal condition of a monitored animal within a given time duration of a given time period, in a prediction stage 110, by providing a monitored record 120 for the monitored animal. The monitored record 120 for the monitored animal can be formed as detailed earlier herein, inter alia with reference to FIGS. 1 and 2. In some cases, the monitored record 120 can include a monitored temperature time series of monitored temperature values that are indicative of a temperature of the monitored animal over the given time period (block 304). In some cases, the monitored record 120 can also include a monitored acceleration time series of monitored acceleration values that are indicative of an acceleration of the monitored animal over the given time period. In some cases, the given time period can be between two and five days. In some cases, the given time duration can be two months or less. In some cases, the given time duration can be one month or less.

In some cases, prediction system 100 can be further configured to smooth the monitored record 120 to reduce an effect of random noise on the monitored temperature values, e.g. using a Kalman filter, thereby providing a smoothed monitored record including a smoothed monitored temperature time series of smoothed monitored temperature values (block 308). In some cases, the monitored record can also be smoothed to reduce an effect of random noise on the monitored acceleration values, if any, e.g. using the Kalman filter, thereby providing a smoothed monitored record including a smoothed monitored temperature time series and a smoothed monitored acceleration time series of smoothed monitored acceleration values.

Prediction system 100 can be configured to analyze the monitored temperature time series (e.g., smoothed monitored temperature time series, unsmoothed monitored temperature time series) of the monitored record (e.g., smoothed monitored record, unsmoothed monitored record), using a Machine Learning (ML) model 130) (block 312), the ML model being trained as detailed earlier herein, inter alia with reference to FIG. 1. In some cases, the ML model can be a Switching Autoregressive Hidden Markov Model (SAR-HMM).

In some cases, in which the ML model 130 is a combined ML model, as defined earlier herein, inter alia with reference to FIG. 1, prediction system 100 can be configured to analyze both a monitored temperature time series (e.g., a smoothed monitored temperature time series or an unsmoothed monitored temperature time series) and a monitored acceleration time series (e.g., a smoothed monitored acceleration time series or an unsmoothed monitored acceleration time series) of the monitored record, using the ML model 130.

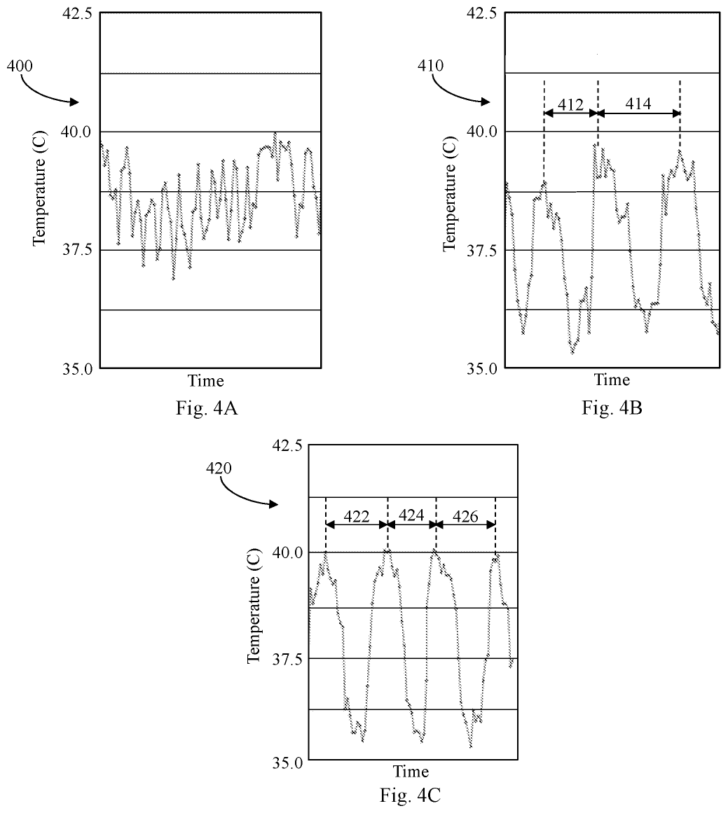

Prediction system 100 can be configured to predict the illness, death or other abnormal condition of the monitored animal within a given time duration of the given time period, based on the analysis, wherein the monitored temperature time series is a near-sinusoidal temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley, as detailed further herein, inter alia with reference to FIGS. 4B and 4C (block 316). In some cases, in which the monitored record includes a monitored temperature time series and does not include a monitored acceleration time series, the illness, death or other abnormal condition of the monitored animal can be predicted, based on the analysis of the monitored temperature time series of the monitored record 120, using the ML model 130, and, optionally, one or more temperature-based meta-heuristics, as detailed below. In some cases, in which the monitored record includes both a monitored temperature time series and a monitored acceleration time series, the illness, death or other abnormal condition of the monitored animal can be predicted, based on the analysis of the monitored temperature time series and the monitored acceleration time series of the monitored record 120, using a combined ML model 130, and, optionally, one or more temperature-based meta-heuristics, as detailed below: Alternatively, in some cases, a temperature-based prediction of the illness, death or other abnormal condition of the monitored animal can be provided based on the analysis using the ML model, and, optionally, one or more temperature-based meta-heuristics, but a final prediction of the illness, death or other abnormal condition of the monitored animal requires both the temperature-based prediction and an acceleration-based prediction of the illness, death or other abnormal condition of the monitored animal, as detailed further herein, inter alia with reference to FIG. 5.

In some cases, a temperature-based prediction or a final prediction of the illness, death or other abnormal condition of the monitored animal within the given time duration of the given time period can be indicative of, for each cycle of the cycles in the monitored temperature time series for the monitored animal over the given time period, a temperature difference between a peak temperature value of the monitored temperature values (e.g., monitored temperature values, smoothed monitored temperature values) in the respective cycle and a valley temperature value of the monitored temperature values in the respective cycle that is greater than or equal to a predetermined difference. In some cases, the predetermined difference is at least 4° C. for each cycle of the cycles, as illustrated in FIG. 4C.

As noted above, in some cases, a temperature-based prediction or a final prediction of the illness, death or other abnormal condition of the monitored animal within a given time duration of the given time period can be provided only if one or more temperature-based meta-heuristics that are associated with the monitored temperature time series (e.g., monitored temperature time series, smoothed monitored temperature time series) for the monitored animal over the given time period are met. In some cases, one of the meta-heuristics is that the peak temperature value for a predefined number of the cycles within the monitored temperature time series is greater than or equal to a temperature threshold. In order to determine if this meta-heuristic is met, prediction system 100, e.g. using server processing circuitry 146, can be configured to determine, for each cycle of the cycles in the monitored temperature time series, whether the peak temperature value for the respective cycle is greater than or equal to a temperature threshold. In some cases, the temperature threshold can be between 39° C. and 41° C. (e.g., 39° C., 39.25° C., 39.5° C., 39.75° C., 40° C., 40.25° C., 40.5° C., 40.75° C., 41° C.). In some cases, the predefined number of cycles can be two or more. In some cases, the predefined number of cycles can be three or more.

In some cases, prediction system 100 can be configured to notify a user, via a user device (e.g., 250-a, . . . , 250-n), of the prediction of (i.e., the final prediction of) the illness, death or other abnormal condition of the monitored animal within the given time duration of the given time period.

Attention is now drawn to FIGS. 4A to 4C, being graphs of monitored temperature time series of monitored records, in accordance with the presently disclosed subject matter.

In accordance with the presently disclosed subject matter, a temperature-based prediction (which, in some cases, can be a final prediction) of an illness, death or other abnormal condition of a monitored animal based on a monitored record for the monitored animal is indicative of the monitored temperature time series of the monitored record being a near-sinusoidal temperature pattern that includes two or more cycles, as detailed earlier herein, inter alia with reference to FIG. 3.

FIG. 4A illustrates a first graph 400 of a first monitored temperature time series of a first monitored record. The first monitored temperature time series is not a temperature pattern that includes two or more cycles, as defined above, and accordingly is not predictive of an illness, death or other abnormal condition of the monitored animal that is associated with the first monitored temperature time series.

FIG. 4B illustrates a second graph 410 of a second monitored temperature time series of a second monitored record. The second monitored temperature time series is a temperature pattern that includes a first cycle 412 and a second cycle 414, as defined above, and accordingly may be predictive of an illness, death or other abnormal condition of the monitored animal that is associated with the second monitored temperature time series, depending, at least in part, on the output of the ML model 130.

FIG. 4C illustrates a third graph 420 of a third monitored temperature time series of a third monitored record. The third monitored temperature time series is a temperature pattern that includes a third cycle 422, a fourth cycle 424 and a fifth cycle 426, as defined above, wherein a temperature difference between the peak temperature in each of the cycles and the valley temperature in each of the cycles is greater than 4° C. Accordingly, the third monitored temperature series may be predictive of an illness, death or other abnormal condition of the monitored animal that is associated with the third monitored temperature time series, depending, at least in part, on the output of the ML model 130.

Attention is now drawn to FIG. 5, a flowchart illustrating a second example of a sequence of operations 500 for predicting an illness, death or other abnormal condition of a monitored animal, in accordance with the presently disclosed subject matter.

In accordance with the presently disclosed subject matter, prediction system 100 can be configured to predict an illness, death or other abnormal condition of a monitored animal within a given time duration of a given time period, in a prediction stage 110, by providing a monitored record for the monitored animal. The monitored record for the monitored animal can be formed as detailed earlier herein, inter alia with reference to FIGS. 1 and 2. In some cases, the monitored record can include a monitored acceleration time series of monitored acceleration values over a given time period, the given time period including a plurality of identical and consecutive sub-periods, each monitored acceleration value of the monitored acceleration values being indicative of an acceleration of the monitored animal over a respective sub-period of the sub-periods (block 504). In some cases, the given time period can be between two to five days. Each of the sub-periods can be of any duration within the given time period, for example a duration of one minute, five minutes, 10 minutes, 15 minutes, 30 minutes, one hour, multiple hours, etc. In some cases, the monitored record can also include a monitored temperature time series of monitored temperature values that are indicative of a temperature of the monitored animal over the given time period.

In some cases, prediction stage 100 can be configured to smooth the monitored record, e.g. using a Kalman filter, to reduce an effect of noise on the monitored acceleration values, thereby providing a smoothed monitored record including a smoothed monitored acceleration time series of smoothed monitored acceleration values (block 508). In some cases, the monitored record can also be smoothed to reduce an effect of random noise on the monitored temperature values, if any, e.g. using the Kalman filter, thereby providing a smoothed monitored record including a smoothed monitored temperature time series and a smoothed monitored acceleration time series.

In some cases, as detailed earlier herein, inter alia with reference to FIG. 1, prediction system 100 can be configured to analyze the monitored record (e.g., monitored record, smoothed monitored record), using a ML model (e.g., a combined ML model). Based on this analysis, prediction system 100 can be configured to predict the illness, death or other abnormal condition of the monitored animal within a given time duration of the given time period associated with the monitored record.

Alternatively, in some cases, prediction system 100 can be configured to determine the monitored acceleration values (e.g., smoothed monitored acceleration values, unsmoothed monitored acceleration values) in the monitored acceleration time series (e.g., smoothed 10 monitored acceleration time series, unsmoothed monitored acceleration time series) that are less than or equal to an acceleration threshold (block 512). In some cases, the acceleration threshold can be constant for all of the monitored acceleration values in a monitored acceleration time series, the constant acceleration threshold being determined, for example, based on historical acceleration values of historical records 140 that are associated with the monitored animal or a group of animals that share one or more characteristics with the monitored animal. Additionally, or alternatively, in some cases, the acceleration threshold can vary across the monitored acceleration time series, for example, based on historical acceleration values of historical records 140 that are associated with the monitored animal or a group of animals that share one or more characteristics with the monitored animal. For example, the acceleration threshold can vary across the monitored acceleration time series, in accordance with a time of day that is associated with each monitored acceleration value of the monitored acceleration values, based on historical acceleration values of the historical records 140. For example, monitored acceleration values that are based on raw acceleration readings that are read from 12 AM to 6 AM can be compared to a first acceleration threshold and monitored acceleration values that are based on raw acceleration readings that are read from 6 AM to 12 PM can be compared to a second threshold, wherein the first acceleration threshold is based on historical acceleration values that are based on raw acceleration readings that are read from 12 AM to 6 AM, and wherein the second acceleration threshold is based on historical acceleration values that are based on raw acceleration readings that are read from 6 AM to 12 PM. Moreover, in some cases, the monitored acceleration values in different monitored records can be compared to different acceleration thresholds, based on historical data regarding historical acceleration values. For example, monitored acceleration values for monitored animals that are associated with a first group of animals (e.g., the monitored animals are present at a first location) can be compared to a first acceleration threshold(s), in accordance with historical acceleration values for animals that are associated with the first group of animals. Moreover, monitored acceleration values for monitored animals that are associated with a second group of animals (e.g., the monitored animals are present at a second location) can be compared to a second acceleration threshold(s), in accordance with historical acceleration values for animals that are associated with the second group of animals. The foregoing examples that describe how the acceleration thresholds for monitored records 120 can be determined are provided for illustrative purposes only, and are not intended to be limiting.

Prediction system 100 can be configured to predict the illness, death or other abnormal condition of the monitored animal within the given time duration of the given time period, based upon a determination that at least a predefined percentage of the monitored acceleration values (e.g., smoothed monitored acceleration values) in the monitored record associated with the monitored animal are less than or equal to the acceleration threshold (block 516). In some cases, the at least a predefined percentage of the monitored acceleration values can be all of the monitored acceleration values. In some cases, the given time period can be two to five days. In some cases, the given time duration can be two months or less. In some cases, the given time duration can be one month or less.

In some cases, in which each of the records (e.g., monitored records 120, historical records 140) includes an acceleration time series (e.g., monitored acceleration time series, historical acceleration time series) and does not include a temperature time series (e.g., monitored acceleration time series, historical acceleration time series), the illness, death or other abnormal condition of the monitored animal can be predicted based solely on the determination that at least a predefined percentage of the monitored acceleration values in the monitored record associated with the monitored animal are less than or equal to the acceleration threshold. Alternatively, in some cases, in which each of the records includes both a temperature time series and an acceleration time series, prediction system 100 can be configured to provide a temperature-based prediction of the illness, death or other abnormal condition of the monitored animal within the given time duration of the given time period, based upon an analysis of the monitored record (e.g., the smoothed monitored record) that is associated with the monitored animal, using a ML model, and optionally other meta-heuristics, as detailed earlier herein, inter alia with reference to FIG. 3. Prediction system 100 can be configured to provide an acceleration-based prediction of the illness, death or other abnormal condition of the monitored animal within the given time duration of the given time period, based upon the determination that at least a predefined percentage of the monitored acceleration values (e.g., smoothed monitored acceleration values) in the monitored record (e.g., smoothed monitored record) that is associated with the monitored animal are less than or equal to an acceleration threshold, as detailed above. Prediction system 100 can be configured to predict (i.e., provide a final prediction of) the illness, death or other abnormal condition of the monitored animal within the given time duration of the given time period, upon the prediction system 100 providing both the temperature-based prediction and the acceleration-based prediction of the illness, death or other abnormal condition of the monitored animal.

In some cases, prediction system 100 can be configured to notify a user, via a user device (e.g., 250)-a, . . . , 250-n), of the prediction of the illness, death or other abnormal condition of the monitored animal within the given time duration of the given time period (block 520).

It is to be noted that, with reference to FIGS. 3 and 5, some of the blocks can be integrated into a consolidated block or can be broken down to a few blocks and/or other blocks may be added. Furthermore, in some cases, the blocks can be performed in a different order than described herein. It is to be further noted that some of the blocks are optional. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A system for predicting an illness, death or other abnormal condition of a monitored animal, the system comprising a processing circuitry configured to:
provide a monitored record for the monitored animal, the monitored record including a monitored temperature time series of monitored temperature values that are indicative of a temperature of the monitored animal over a given time period;
analyze the monitored temperature time-series;
predict the illness, death or other abnormal condition of the monitored animal within a given time duration of the given time period, based on the analysis, wherein the prediction is indicative of the monitored temperature time-series is being a temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley; and
notify a user of the system of the prediction.

2. The system of claim 1, wherein the processing circuitry is configured to analyze the monitored temperature time series using a Machine Learning (ML) model, the ML model being trained based on a data repository of historical records for a plurality of animals, each historical record of the historical records including: (A) a historical temperature time series of historical temperature values that are indicative of the temperature of a respective animal of the plurality of animals over an earlier time period, being earlier than and of an identical duration to the given time period, and (B) a target field that indicates whether the respective animal became ill, died, or developed any other abnormal condition within the given time duration of the earlier time period.

3. (canceled)

4. The system of claim 1, wherein, for each cycle of the cycles, a temperature difference between a peak temperature value of the monitored temperature values in the respective cycle and a valley temperature value of the monitored temperature values in the respective cycle is greater than or equal to a predetermined difference.

5. The system of claim 4, wherein the predetermined difference is at least 4° C.

6. The system of claim 1, wherein the given time period is two to five days.

7. The system of claim 1, wherein the given time duration is two months or less.

8. (canceled)

9. The system of claim 1, wherein the processing circuitry is further configured to:
determine, for each cycle of the cycles, whether the peak temperature value for the respective cycle is greater than or equal to a temperature threshold;
wherein the prediction is indicative of a number of the cycles for which the peak temperature value is greater than or equal to the temperature threshold being greater than or equal to a predefined number.

10. (canceled)

11. (canceled)

12. The system of claim 1, wherein the monitored record includes a monitored acceleration time series of monitored acceleration values over the given time period, the given time period including a plurality of identical and consecutive sub-periods, and each monitored acceleration value of the monitored acceleration values being indicative of an acceleration of the monitored animal over a respective sub-period of the sub-periods, wherein the processing circuitry is further configured to:
determine the monitored acceleration values in the monitored acceleration time-series that are less than or equal to an acceleration threshold; and
wherein the predict is also based on a determination that at least a predefined percentage of the monitored acceleration values are less than or equal to the acceleration threshold.

13. (canceled)

14. The system of claim 12, wherein the processing circuitry is further configured to:
provide historical acceleration values for one or more animals, each historical acceleration value of the historical acceleration values being indicative of the acceleration of a respective animal of the one or more animals over a second respective sub-period, being earlier than and of an identical duration to the respective sub-period; and
determine the acceleration threshold, based on the historical acceleration values.

15. (canceled)

16. A method for predicting an illness, death or other abnormal condition of a monitored animal, the method comprising:
providing a monitored record for the monitored animal, the monitored record including a monitored temperature time series of monitored temperature values that are indicative of a temperature of the monitored animal over a given time period;
analyzing the monitored temperature time series;
predicting the illness, death or other abnormal condition of the monitored animal within a given time duration of the given time period, based on the analysis, wherein the prediction is indicative of the monitored temperature time series is being a temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley; and
notifying a user of the prediction.

17. The method of claim 16, wherein the monitored temperature time series is analyzed using a Machine Learning (ML) model, the ML model being trained based on a data repository of historical records for a plurality of animals, each historical record of the historical records including: (A) a historical temperature time series of historical temperature values that are indicative of the temperature of a respective animal of the plurality of animals over an earlier time period, being earlier than and of an identical duration to the given time period, and (B) a target field that indicates whether the respective animal became ill, died, or developed any other abnormal condition within the given time duration of the earlier time period.

18. (canceled)

19. The method of claim 16, wherein, for each cycle of the cycles, a temperature difference between a peak temperature value of the monitored temperature values in the respective cycle and a valley temperature value of the monitored temperature values in the respective cycle is greater than or equal to a predetermined difference.

20. The method of claim 19, wherein the predetermined difference is at least 4° C.

21. The method of claim 16, wherein the given time period is two to five days.

22. The method of claim 16, wherein the given time duration is two months or less.

23. (canceled)

24. The method of claim 16, further comprising:
determining, for each cycle of the cycles, whether the peak temperature value for the respective cycle is greater than or equal to a temperature threshold;
wherein the prediction is indicative of a number of the cycles for which the peak temperature value is greater than or equal to the temperature threshold being greater than or equal to a predefined number.

25. The method of claim 24, wherein the temperature threshold is between 39° C. and 41° C.

26. (canceled)

27. The method of claim 16, wherein the monitored record includes a monitored acceleration time series of monitored acceleration values over the given time period, the given time period including a plurality of identical and consecutive sub-periods, and each monitored acceleration value of the monitored acceleration values being indicative of an acceleration of the monitored animal over a respective sub-period of the sub-periods, wherein the method further comprises:
determining the monitored acceleration values in the monitored acceleration time-series that are less than or equal to an acceleration threshold;
wherein the predicting is also based on a determination that at least a predefined percentage of the monitored acceleration values are less than or equal to the acceleration threshold.

28. (canceled)

29. The method of claim 27, further comprising:
providing historical acceleration values for one or more animals, each historical acceleration value of the historical acceleration values being indicative of the acceleration of a respective animal of the one or more animals over a second respective sub-period, being earlier than and of an identical duration to the respective sub-period; and
determining the acceleration threshold, based on the historical acceleration values.

30. (canceled)

31. A non-transitory computer readable storage medium having computer readable program code embodied therewith, the computer readable program code, executable by a processing circuitry of a computer to perform a method for predicting an illness, death or other abnormal condition of a monitored animal, the method comprising:
providing a monitored record for the monitored animal, the monitored record including a monitored temperature time series of monitored temperature values that are indicative of a temperature of the monitored animal over a given time period;
analyzing the monitored temperature time series;
predicting the illness, death or other abnormal condition of the monitored animal within a given time duration of the given time period, based on the analysis, wherein the prediction is indicative of the monitored temperature time series being a temperature pattern that includes two or more cycles, each cycle of the cycles being defined by a distance between a given peak of the temperature pattern and a successive peak of the temperature pattern, successive to the given peak, or alternatively, a distance between a given valley of the temperature pattern and a successive valley of the temperature pattern, successive to the given valley; and
notifying a user of the prediction.
